SEQUENCE LISTING (6 sequences)

SEQ ID NO: 1 - protein, 107 aa, synthetic construct
  REGION 1..107: note = Light chain variable amino acid sequence: 107 amino acids
DIQMTQSPSSLSASVGDRVTITCKASRDIRSYLTWYQQKPGKAPKTLIYYATSLADGVPSRFSGSGSGQDYSLTISSLESDDTATYYCLQHGESPFTLGSGTKLEIK

SEQ ID NO: 2 - protein, 119 aa, synthetic construct
  REGION 1..119: note = Heavy chain variable amino acid sequence: 119 amino acids
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSS

SEQ ID NO: 3 - DNA, 357 bp, synthetic construct
  misc_feature 1..357: note = Heavy chain variable nucleotide sequence
  source 1..357: mol_type = other DNA
gaagtgcagctggtggagtctgggggaggcttagtgaagcctggagggtccctgaaactctcctgtgcagcctctggattcaagtttagtagatatgccatgtcttgggttcgccaggctccggggaagaggctggagtgggtcgcaaccattagtagtggtggtagttacatctactatccagacagtgtgaagggtcgattcaccatctccagagacaatgtcaagaacaccctgtatctgcaaatgagcagtctgaggtctgaggacacggccatgtattactgtgcaagacgagattacgacctggactactttgactcctggggccaaggcacccttgtcaccgtctcctca

SEQ ID NO: 4 - DNA, 321 bp, synthetic construct
  misc_feature 1..321: note = Light chain variable nucleotide sequence
  source 1..321: mol_type = other DNA
gacatccagatgacccagtctccatcctccctgtctgcatcggtgggagacagagtcactatcacttgcaaggcgagtcgggacattagaagctatttaacctggtaccagcagaaaccagggaaagctcctaagaccctgatctattatgcaacaagcttggcagatggggtcccgtcgagattcagtggcagtggatctgggcaagattattctctcaccatcagcagcctggagtctgacgatacagcaacttactactgtctacaacatggtgagagtccattcacgctcggctcggggaccaagctggaaatcaaa

SEQ ID NO: 5 - protein, 449 aa, synthetic construct
  REGION 1..449: note = Heavy chain full amino acid sequence
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, synthetic construct
  REGION 1..214: note = Light chain full amino acid sequence
DIQMTQSPSSLSASVGDRVTITCKASRDIRSYLTWYQQKPGKAPKTLIYYATSLADGVPSRFSGSGSGQDYSLTISSLESDDTATYYCLQHGESPFTLGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0101700 A1
(43) Pub. Date: Mar. 28, 2024

(54) USE OF ITOLIZUMAB TO REDUCE PHOSPHORYLATION OF CD6

(71) Applicant: Biocon Limited, Bangalore (IN)

(72) Inventors: Pradip NAIR, Bangalore (IN); Arindam SAHA, Bangalore (IN); Ravindra Belavinakodige SADASHIVARAO, Karnataka (IN); Usha BUGHANI, Bangalore (IN); Ramakrishnan MELARKODE, Bangalore (IN)

(21) Appl. No.: 18/335,542

(22) Filed: Jun. 15, 2023

Related U.S. Application Data
(63) Continuation of application No. 16/387,442, filed on Apr. 17, 2019, now abandoned.
Application No. 16/387,442 is a continuation of application No. PCT/IB2017/056403, filed on Oct. 16, 2017.

(30) Foreign Application Priority Data
Oct. 18, 2016 (IN) 201641035602

(51) Int. Cl. C07K 16/28; A61K 39/00; A61P 19/02; A61P 29/00; A61P 35/00; A61P 37/06

(52) U.S. Cl. CPC C07K 16/2896; A61K 39/0008; A61P 19/02; A61P 29/00; A61P 35/00; A61P 37/06; C07K 2317/24; C07K 2317/32; C07K 2317/76

(57) ABSTRACT

The present invention discloses a key mechanism of action of itolizumab that involves a decrease in an activating ALCAM-CD6 co stimulatory signal by directly reducing CD6 hyperphosphorylation and preventing the docking of key molecules associated with T cell activation and signaling.

### CROSS-REFERENCE TO RELATED APPLICATION

The present application is a continuation application of U.S. application Ser. No. 16/387,442, filed Apr. 17, 2019, which is a continuation application of international application No. PCT/IB2017/056403, filed Oct. 16, 2017, which claims the benefit of and the priority to provisional Indian patent application 201641035602 filed on 18 Oct. 2016 with the Indian Patent Office. The content of said application filed on 18 Oct. 2016 is incorporated herein by reference for all purpose in its entirety, including an incorporation of any element or part of the description, claims or drawings not contained herein and referred to in Rule 20.5(a) of the PCT, pursuant to Rule 4.18 of the PCT.

### STATEMENT REGARDING THE SEQUENCE LISTING

The Sequence Listing XML associated with this application is provided in XML file format and is hereby incorporated by reference into the specification. The name of the XML file containing the Sequence Listing XML is EQIL_007_02 US_ST26.xml. The XML file is 10,205 bytes, created on Jun. 14, 2023, and is being submitted electronically via USPTO Patent Center.

### FIELD OF THE INVENTION

The present invention relates to a humanized IgG1 isotype anti-CD6 monoclonal antibody (T1h) that binds to the scavenger receptor cysteine-rich (SRCR) domain 1(D1) of CD6 present on the surface of thymic epithelial cells, monocytes, activated T-cells and a variety of other cells types. The present invention relates to method for treatment, including prevention of disease conditions mediated by T-helper and T lymphocytes cells.

### BACKGROUND OF THE INVENTION

T-cell activation, differentiation and function is controlled by co-stimulatory and co-inhibitory receptors with diverse expression, structure and function, and is largely context dependent. The activation of TCR and subsequent phosphorylation of ZAP70 facilitated CD6 association to the TCR complex where CD6 acts like a scaffold protein permitting the recruitment of SLP-76 and the guanine nucleotide factor Vav1 independent of LAT, an adaptor or docking protein (Roncagalli R et al, 2016)). In addition hyper phosphorylation at tyrosine, serine and threonine residues on the cytoplasmic tail of CD6, leads to CD6 binding to adaptor molecules such as SLP-76 followed by time and dose dependent MAPK activation (Nair P et al, 2010). CD6 was identified as a signaling attenuator whose expression alone, i.e. even in the absence of ligand engagement was sufficient to restrain signaling in T-cells (Oliveira L et al, 2012). Further recently, Orta-Mascaro M et al., 2016 have shown that in CD6 null mice there is a negative selection in thymus and an increased activation in response to self- or environmental antigens in the periphery. This finding is indicated by an expansion of T cell subsets with memory and regulatory phenotypes, indicating an inhibitory function for CD6.

CD6 is associated with T-cell modulation and is implicated in several autoimmune diseases. WO/2009/113083 showed that a humanized IgG1 isotype anti-CD6 antibody (T1h) that binds to the scavenger receptor cysteine-rich (SRCR) domain 1(D1) of CD6 present on the surface of thymic epithelial cells, monocytes, activated T cells and a variety of other cells types. CD6 and CD5, both being members of the scavenger receptor cysteine rich domain superfamily (SRCR-SF) and sharing considerable structural and functional homology, were individually found to be superior than classical CD28 mediated co-stimulation with anti-CD3 to prime naive T-cells to differentiate into Th17 cell.

WO/2015/011658 demonstrated that itolizumab, a CD6 domain 1 specific humanized monoclonal antibody, inhibited the proliferation and cytokine production of T lymphocytes stimulated with anti-CD3 antibody or when co-stimulated with ALCAM. Itolizumab also has demonstrated efficacy in human diseases known to have an IL-17 driven pathogenesis.

Itolizumab is a humanized IgG1 non-depleting monoclonal antibody (mAb) which binds to domain 1 of CD6 without interfering with ALCAM and CD6 domain 3 binding. Recent clinical trials with itolizumab have demonstrated efficacy in psoriasis and rheumatoid arthritis patients, and this drug has been approved for treatment of psoriasis in India (Krupashankar D S et al, 2014). However, the mode of action of this drug is not clearly understood. Thus it would be advantageous to discover the mode of action for itolizumab.

### SUMMARY OF THE INVENTION

The present invention discloses that a key mechanism of action of itolizumab, which involves a decrease in an activating ALCAM-CD6 co stimulatory signal by directly reducing CD6 hyper phosphorylation and preventing the docking of key molecules associated with T cell signaling, activation and proliferation.

In one aspect, the present invention provides a method of reducing phosphorylation of a CD6-ALCAM complex, the method comprising:
contacting a host cell with a monoclonal anti-CD6 antibody comprising heavy and light chain variable regions as set forth in SEQ ID NO: 1 and 2 respectively, wherein the binding of the monoclonal anti-CD6 antibody to D1 receptor on CD6 causes a steric hindrance for interaction of activated leukocyte cell adhesion molecule (ALCAM) with D3 receptor of CD6, thereby causing a reduction of phosphorylation of CD6 receptor of the CD6-ALCAM complex. Preferably, the monoclonal anti-CD6 is itolizumab.

Importantly, the reduction of phosphorylation of CD6 of the CD6-ALCAM complex also causes a reduction in docking of ZAP 70 (cytoplasmic protein tyrosine kinase that plays a critical role initiating T-cell responses) and SLP-76 (a docking molecule) thereby reducing the expression of phosphatases SHP1 and SHP2.

In yet another aspect, the present invention provides for a method inhibiting full interaction of the formed CD6-ALCAM complex due to steric hindrance at the immunological synapse, the method comprising:
contacting a host cell with a monoclonal anti-CD6 antibody comprising heavy and light chain variable regions as set forth in SEQ ID NO: 1 and 2 respectively, wherein the binding of the monoclonal anti-CD6 antibody to D1 receptor on CD6 causes a steric hindrance for interaction of ALCAM with D3 receptor of CD6, thereby causing a reduction of phosphorylation of CD6 receptor of the CD6-ALCAM complex. Preferably, the monoclonal anti-CD6 is itolizumab.

In a further aspect, the present invention provides for reduction of phosphorylation of a CD6 receptor induced by binding of ALCAM to D3 of CD6, the method comprising:
contacting a host cell with an anti-CD6 antibody that binds to D1 of the CD6, wherein the anti-CD6 antibody comprises a heavy and light chain variable regions as set forth in SEQ ID NO: 1 and 2 respectively, wherein the binding of the monoclonal anti-CD6 antibody to D1 receptor on CD6 causes causing a reduction of phosphorylation of CD6 receptor of a CD6-ALCAM complex.

In a still further aspect, the present invention provides for a method of inhibiting expression of phosphatases SHP1 and SHP2, the method comprising:
contacting a host cell with a monoclonal anti-CD6 antibody comprising heavy and light chain variable regions as set forth in SEQ ID NO: 1 and 2 respectively, wherein the binding of the monoclonal anti-CD6 antibody to D1 receptor on CD6 causes a reduction of phosphorylation of CD6 receptor of the CD6-ALCAM complex, thereby reducing the expression of phosphatases SHP1 and SHP2.

The host cell in the above discussed methods is preferably in a human subject in need of treatment for modulating inflammatory conditions like psoriasis, rheumatoid arthritis or autoimmune responses in patients like adverse responses associated with multiple sclerosis or transplant rejection, graft-versus-host disease, type-1 and type-2 diabetes, cutaneous T cell lymphoma, thyroditis and other T cell mediated autoimmune diseases.

Other aspects, objects, features and advantages of the present invention would be apparent to one of ordinary skill in the art from the following detailed description illustrating the preferred embodiments of the invention.

### BRIEF DESCRIPTION OF FIGURES

FIG. 1 shows that itolizumab inhibits CD6-ALCAM co-stimulatory signal transduction pathway. Human PBMCs were plated on ALCAM (10 μg/ml) coated plates for 40 minutes with itolizumab, or Iso Ab. (A) CD6 was immune precipitated with either itolizumab or Iso Ab and immune blotted for CD6, p-Tyr, Zap70 and SLP-76 (top panels). Bottom panels show the corresponding 10% input control samples for CD6, ZAP70, and SLP-76. Representative blots are from at least three independent experiments from different donors. (B-D) Overall quantification of p-Tyr, Zap70 and SLP-76 intensity as represented in FIG. A, from three independent experiments are shown as a bar graphs. Results are expressed as mean±SD. (E) Similar experiment as described in (A), is now immune blotted for CD6, p-Tyr, and for phosphatases p-SHP1, SHP1, pSHP2 and SHP2. These blots are representative of three independent experiments from different donors. (F and G) Overall quantification of p-SHP1 and p-SHP2 intensity as represented in figure E, from three independent experiments is shown as bar graphs. Results are expressed as mean±SD.

FIG. 2 shows the CD6 western blot of CD6 immune precipitated samples using MEM-98 antibody.

FIG. 3 shows (A) human PBMCs were treated with 0.5 ng/ml anti-CD3 antibody (OKT3) for 24 h in presence or absence of itolizumab or Iso Ab. CD6 was immune precipitated with itolizumab and immune blotted for CD6, p-Tyr, Zap70 and SLP-76. Corresponding 10% input samples were run as negative controls. Representative blots are from two independent experiments. (B-D) Quantification (mean+SD) of p-Tyr, Zap70, SLP-76 relative intensity. Graphs are drawn from two independent experiments to calculate the fold difference in different experimental conditions.

FIG. 4 shows a cartoon depicting the proposed mechanism of action of itolizumab. Here it is shown that ALCAM-CD6 optimum interaction is inhibited by steric hindrance caused by itolizumab. Inhibition of ALCAM-CD6 interaction decreases CD6 phosphorylation in its cytoplasmic domain leading to down-regulation of T cell activation signaling cascade.

FIG. 5 shows the light variable amino acid sequence (SEQ ID NO: 1), heavy variable amino acid sequence (SEQ ID NO: 2), heavy variable and constant amino acid sequence (SEQ ID NO: 5) and light variable and constant amino acid sequence (SEQ ID NO: 6) of the itolizumab antibody.

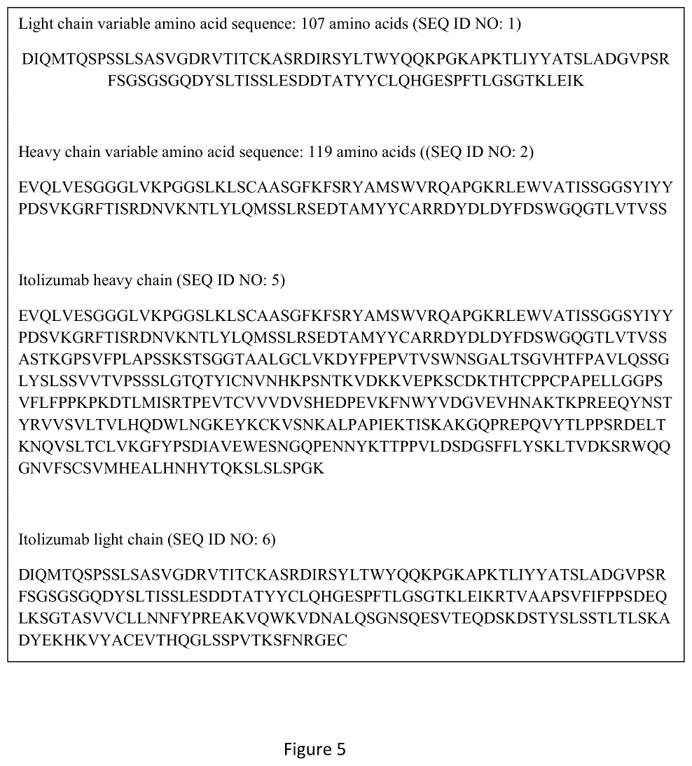

### DETAILED DESCRIPTION OF THE INVENTION

The present invention provides for an anti-CD6 monoclonal antibody capable of binding to domain 1(D1) of CD6 and directly inhibits or reduces CD6 receptor phosphorylation induced by ALCAM and subsequent decrease in docking of associated ZAP70 (a kinase) and docking protein SLP76. Further, such inhibition and/or reduction in CD6 phosphorylation and associated signaling molecules leads to decreased T-cell activation and differentiation.

The practice of the present invention will employ, unless otherwise indicated, conventional techniques of immunology, molecular biology, microbiology, cell biology and recombinant DNA, which are within the skill of the art. See, e.g., Sambrook, et al. MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular.

In describing and claiming the present invention, the following terminology will be used in accordance with the definitions set out herein.

As used herein, “anti-CD6 antibody” is generally an antibody that bind specifically to SRCR domain 1 (D1) of human CD6 (hCD6). In preferred aspects of the invention, antibodies and other immunoglobulins, including native and artificially modified antibodies and antibody fragments, are provided that bind specifically to human SRCR domain 1 of CD6 and that do not interfere with the activated leukocyte cell adhesion molecule (ALCAM) binding to CD6.

As used herein, “monoclonal antibody” (mAb) refers to an antibody of a population of substantially homogeneous antibodies; that is, the individual antibodies in that population are identical except for naturally occurring mutations that may be present in minor amounts. Monoclonal antibodies are highly specific, being directed against a single antigenic determinant, an “epitope.” Therefore, the modifier “monoclonal” is indicative of a substantially homogeneous population of antibodies directed to the identical epitope and is not to be construed as requiring production of the antibody by any particular method. It should be understood that monoclonal antibodies can be made by any technique or methodology known in the art; including e.g., recombinant DNA methods known in the art, or methods of isolation of monoclonal recombinantly produced using phage antibody libraries.

As used herein, “therapeutically effective amount” refers to an amount effective, at dosages and for periods of time necessary, to achieve a desired therapeutic result.

It is understood that aspects of the present invention described herein also include “consisting of” and “consisting essentially of” aspects.

The present invention provides an anti-CD6 monoclonal antibody that is capable of specifically binding to D1 domain of CD6 without interfering with the binding of ALCAM to CD6 comprising SEQ ID NO: 1 and SEQ ID NO: 2. The nucleotide sequences encoding the anti-CD6 monoclonal antibody includes SEQ ID NO: 3 and SEQ ID NO: 4, respectively or nucleotide sequences have at least 90% identity thereto and encode for SEQ ID NO: 1 and SEQ ID NO: 2.

Methods for Producing the Anti-CD6 Monoclonal Antibodies of the Invention

The present invention further provides methods for producing the disclosed anti-CD6 antibodies. These methods encompass culturing a host cell containing isolated nucleic acid(s) encoding the antibodies of the invention. As will be appreciated by those in the art, this can be done in a variety of ways, depending on the nature of the antibody.

In general, nucleic acids are provided that encode the antibodies of the invention. The polynucleotides can be in the form of RNA or DNA. Polynucleotides in the form of DNA, cDNA, genomic DNA, nucleic acid analogs, and synthetic DNA are within the scope of the present invention. The DNA may be double-stranded or single-stranded, and if single stranded, may be the coding (sense) strand or non-coding (anti-sense) strand. The coding sequence that encodes the an anti-CD6 monoclonal antibody may be identical to the coding sequence provided herein or may be a different coding sequence, which sequence, as a result of the redundancy or degeneracy of the genetic code, encodes the same polypeptides as the DNA provided herein.

In some embodiments, nucleic acid(s) encoding the anti-CD6 monoclonal antibody of the present invention are incorporated into expression vectors, which can be extrachromosomal or designed to integrate into the genome of the host cell into which it is introduced. Expression vectors can contain any number of appropriate regulatory sequences (including, but not limited to, transcriptional and translational control sequences, promoters, ribosomal binding sites, enhancers, origins of replication, etc.) or other components (selection genes, etc.), all of which are operably linked as is well known in the art. In some cases two nucleic acids are used and each put into a different expression vector (e.g. heavy chain in a first expression vector, light chain in a second expression vector), or alternatively they can be put in the same expression vector. It will be appreciated by those skilled in the art that the design of the expression vector(s), including the selection of regulatory sequences may depend on such factors as the choice of the host cell, the level of expression of protein desired, etc.

In general, the nucleic acids and/or expression can be introduced into a suitable host cell to create a recombinant host cell using any method appropriate to the host cell selected (e.g., transformation, transfection, electroporation, infection), such that the nucleic acid molecule(s) are operably linked to one or more expression control elements (e.g., in a vector, in a construct created by processes in the cell, integrated into the host cell genome). The resulting recombinant host cell can be maintained under conditions suitable for expression (e.g. in the presence of an inducer, in a suitable non-human animal, in suitable culture media supplemented with appropriate salts, growth factors, antibiotics, nutritional supplements, etc.), whereby the encoded polypeptide(s) are produced. In some cases, the heavy chains are produced in one cell and the light chain in another.

The expression vectors can be transfected into host cells such as E. coli cells, mammalian cells such as simian COS cells of Chinese hamster ovary (CHO) cells, Bacillus, Streptomyces, and Saccharomyces to obtain the synthesis of monoclonal antibodies in the recombinant host cells. Yeast, insect, and plant cells can also be used to express recombinant antibodies. In some embodiments, the antibodies can be produced in transgenic animals such as cows or chickens.

General methods for antibody molecular biology, expression, purification, and screening are described, for example, in Antibody Engineering, edited by Kontermann & Dubel, Springer, Heidelberg, 2001 and 2010.

Mode of Administration

For administration in the methods of use described below, the anti-CD6 monoclonal antibody may be mixed, prior to administration to a human subject in need of such treatment, with a non-toxic, pharmaceutically acceptable carrier substance (e.g. normal saline or phosphate-buffered saline), and will be administered using any medically appropriate procedure, e.g., parenteral administration (e.g., injection) such as by intravenous or intra-arterial injection.

Formulations of the anti-CD6 monoclonal antibody used in accordance with the present invention may be prepared by mixing an antibody having the desired degree of purity with optional pharmaceutically acceptable carriers, excipients or stabilizers in either the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol and m-cresol; low molecular weight (less than about residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The anti-CD6 monoclonal antibody may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are well known in the art.

Sustained-release preparations may be prepared. Suitable examples of sustained-release preparations include semipermeable matrices of solid hydrophobic polymers containing the anti-CD6 monoclonal antibody, which matrices are in the form of shaped articles, e.g. films, or microcapsules. Examples of sustained-release matrices include polyesters, hydrogels, copolymers of L-glutamic acid, non-degradable ethylene-vinyl acetate and degradable lactic acid-glycolic acid copolymers.

The anti-CD6 monoclonal antibody may be administered to a mammalian such as a human subject in need of treatment, in accord with known methods, such as intravenous administration as a bolus or by continuous infusion over a period of time, by intramuscular, intraperitoneal, intracerobrospinal, subcutaneous, intra-articular, intrasynovial, intrathecal or oral routes. Intravenous or subcutaneous administration of the anti-CD6 monoclonal antibody is preferred.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. The efficient dosages and the dosage regimens for the anti-CD6 monoclonal antibodies used in the present invention depend on the severity of the lupus-type disease and may be determined by the persons skilled in the art.

An exemplary, non-limiting range for a therapeutically effective amount of the anti-CD6 monoclonal antibody used in the present invention is about 0.01-100 mg/kg per subject body weight, such as about 0.01-50 mg/kg, for example about 0.01-25 mg/kg. A medical professional having ordinary skill in the art may readily determine and prescribe the effective amount of the pharmaceutical composition required. For example, a physician could start doses of the anti-CD6 monoclonal antibody at levels lower than that required in order to achieve the desired therapeutic effect and gradually increase the dosage until the desired effect is achieved.

In one embodiment, the anti-CD6 monoclonal antibody is administered by infusion in a weekly dosage of from 1 to 500 mg/kg per subject body weight, such as, from 20 to 200 mg/kg. Such administration may be repeated, e.g., 1 to 8 times, such as 3 to 5 times. In the alternative, the administration may be performed by continuous infusion over a period of from 2 to 24 hours, such as, from 2 to 12 hours.

In another embodiment the anti-CD6 monoclonal antibody is administered in a weekly dosage of from 10 mg to 200 mg, for up to 7 times, such as from 4 to 6 times. The administration may be performed by continuous infusion over a period of from 2 to 24 hours, such as, from 2 to 12 hours. Such regimen may be repeated one or more times as necessary, for example, after 6 months or 12 months.

The Examples which follow are set forth to aid in understanding the invention but are not intended to, and should not be construed to limit its scope in any way. The Examples do not include detailed descriptions for conventional methods employed in the assay procedures. Such methods are well known to those of ordinary skill in the art and are described in numerous publications including by way of examples.

### Examples

Previous studies by the current inventors depicted that addition of itolizumab (SEQ ID NOs: 1-2 encoded by SEQ ID NOs: 3 and 4) binds to domain 1 of CD6 and reduces the activation and differentiation of T cells to Th17 cells and decreases production of IL-17. These effects are associated with the reduction of key transcription factors pSTAT3 and RORγT. In the currents examples, the effect of itolizumab on ALCAM-CD6 mediated T cell activation was evaluated to understand the mechanism of inhibition.

Both monoclonal antibodies, itolizumab and nimotuzumab (humanized anti EGFR, identical Fc region as Itolizumab) mAbs were produced at Biocon Ltd (Bangalore, India) and used in soluble form in all the experiments. Nimotuzumab, was used as a non-specific isotype control antibody in all experiments (Iso Ab).

Itolizumab Inhibits CD6-ALCAM Mediated Co-Stimulatory Signal Transduction Pathway

To understand the physiological basis for itolizumab-mediated inhibition of T cell activation, the role of itolizumab was investigating in inhibiting an activating CD6-ALCAM interaction. To evaluate signal transduction downstream to CD6, PBMCs were treated with and without itolizumab in presence of plate bound ALCAM.

In this experiment, 6 well plates were coated overnight with Fc-ALCAM (10 μg/ml) in TSM buffer (20 mM Tris, 150 mM NaCl, 1 mM CaCl₂), 2 mM MgCl₂, 1× protease and phosphatase inhibitors added). On the day of experiment, coated plates were blocked with 1% BSA in TSM buffer. Human PBMC (5×10⁶/well in a 6 well plate) were plated and treated with itolizumab or Iso Ab for 40 minutes.

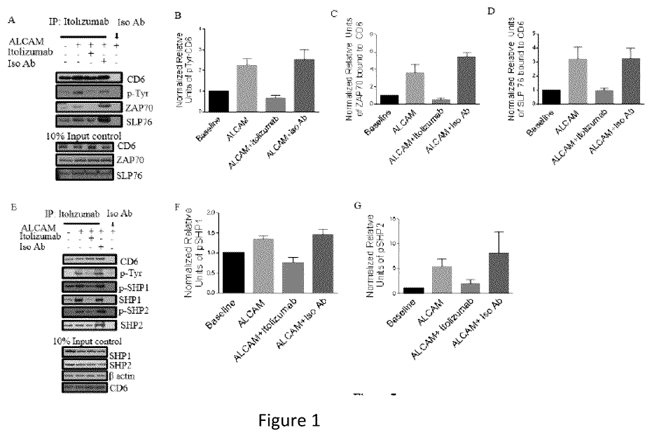

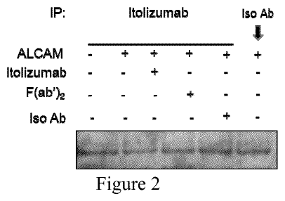

Using this technique, ALCAM dependent CD6 phosphorylation and CD6-interacting molecules from immune-precipitated CD6 protein were investigated. Equal CD6 pull down by itolizumab was confirmed by CD6 immuno blot using 2 different antibodies itolizumab and MEM-98 (FIG. 1 and FIG. 2). Tyrosine phosphorylation of pulled down CD6 protein was investigated, and results showed that ALCAM-CD6 interaction increased CD6 tyrosine phosphorylation by over 2.5 fold. This increase in phosphorylation was inhibited by itolizumab. Zap70 (a Kinase) and SLP-76 (a signaling and/or docking protein), known binding partners of CD6 were examined for their association with immunoprecipitated CD6. ALCAM-CD6 interaction increased SLP-76 and Zap70 association with CD6 by 3-4 fold, and again this was inhibited by itolizumab (FIG. 1).

Phosphorylation of receptors is controlled by expression of phosphatases. SHP1 and SHP2 are key phosphatases known to be associated with receptor proteins and control their phosphorylation thereby modulating signal transduction. The association and phosphorylation of these proteins with immunoprecipitated CD6 was investigated in itolizumab-mediated inhibition. As shown in FIG. 2, the binding complex of ALCAM-CD6 interaction increased phosphorylation of CD6 associated SHP1 and SHP2 by 3-4 fold. However, and surprisingly, the use of itolizumab inhibited both total and phosphorylated (activated) SHP1 and SHP2 associated with CD6 thereby bringing their expression to baseline levels (FIG. 1). These results suggest the inhibition of T cell activation by Itolizumab is not via overexpression or activation of phosphatases but by direct decrease in CD6 hyper phosphorylation independent of SHP1 and SHP2.

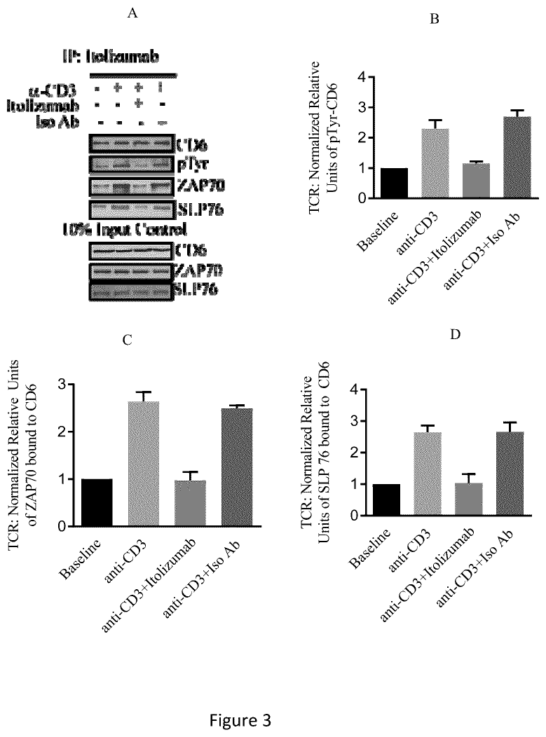

To prove the effect of ALCAM-CD6 in a more physiological relevant condition, the TCR activation experiment was used. In these experiments, PBMC were treated with 0.5 ng/ml anti-CD3 antibody (OKT 3) for 24 h in presence or absence of itolizumab or Iso Ab antibody. Cells were harvested and CD6 was immune-precipitated with itolizumab or Iso Ab. 10% total lysate served as input control samples. Both immune precipitated and input control samples were immune blotted and analyzed. Here the results show that anti CD3-mediated activation increased CD6 phosphorylation, Zap70 and SLP-76 association with CD6 by 2.5-3 fold respectively. In all cases, these activation signals were completely inhibited in presence of itolizumab, as shown in FIG. 3. Overall, these results indicate that a key mechanism of action of itolizumab involves a decrease in an activating ALCAM-CD6 co stimulatory signal by directly reducing CD6 hyper phosphorylation and preventing the docking of key molecules associated with T cell signaling, activation and proliferation.

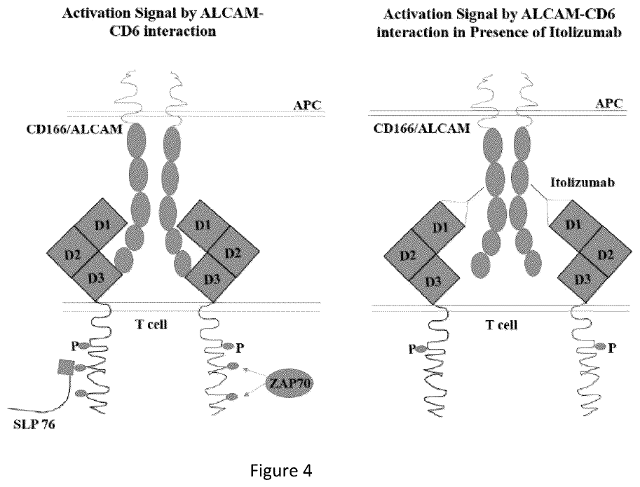

The present invention shows that at the molecular level, itolizumab prevents the optimal engagement of CD6-ALCAM critical for T-cell activation. A theoretical model for such interaction is shown in FIG. 4. Clustering refers to accumulation of CD6 receptors on T cell membrane.

Accumulation (clustering) of CD6 at the immunological synapse initiates activation of CD6 receptor by interacting with ALCAM on antigen presenting cells and thus forming CD6-ALCAM complex. In this model it is suggested that itolizumab, upon binding to domain one of CD6, provides a steric hindrance and prevents the optimal interaction of ALCAM with domain 3 (D3) of CD6. This steric hindrance results in the attenuation of T cell signaling mediated by this costimulatory molecule CD6. Under other circumstances, where CD6 is not clustered, itolizumab does not prevent or interfere with ALCAM-CD6 interaction as was reported earlier. This explains a key mechanism of action of itolizumab by which, an activating ALCAM-CD6 interaction is blocked.

### REFERENCES

The contents of any references cited herein are incorporated by reference herein for all purposes.
Roncagalli R, Hauri S, Fiore F, Liang Y, Chen Z, Sansoni A, et al. Quantitative proteomics analysis of signalosome dynamics in primary T cells identifies the surface receptor CD6 as a Lat adaptor-independent TCR signaling hub. Nat Immunol. 2014 April; 15(4):384-92.
Nair P, Melarkode R, Rajkumar D, Montero E. CD6 synergistic co-stimulation promoting proinflammatory response is modulated without interfering with the activated leucocyte cell adhesion molecule interaction. Clin Exp Immunol. 2010 October; 162(1):116-30.
Oliveira M I, Goncalves C M, Pinto M, Fabre S, Santos A M, Lee S F, et al. CD6 attenuates early and late signaling events, setting thresholds for T-cell activation. Eur J Immunol. 2012 January; 42(1):195-205.
Orta-Mascaro M, Consuegra-Fernandez M, Carreras E, Roncagalli R, Carreras-Sureda A, Alvarez P, et al. CD6 modulates thymocyte selection and peripheral T cell homeostasis. J Exp Med. 2016 Jul. 25; 213(8):1387-97.
Krupashankar D S, Dogra S, Kura M, Saraswat A, Budamakuntla L, Sumathy T K, et al. Efficacy and safety of itolizumab, a novel anti-CD6 monoclonal antibody, in patients with moderate to severe chronic plaque psoriasis: results of a double-blind, randomized, placebo-controlled, phase-III study. J Am Acad Dermatol. 2014 September; 71(3):484-92.

1. A method of reducing phosphorylation of a CD6-ALCAM complex, the method comprising:
contacting a host cell with a monoclonal anti-CD6 antibody comprising heavy and light chain variable regions as set forth in SEQ ID NO: 1 and 2 respectively, wherein the binding of the monoclonal anti-CD6 antibody to D1 receptor on CD6 causes a steric hindrance for interaction of ALCAM with D3 receptor of CD6, thereby causing a reduction of phosphorylation of CD6 receptor of the CD6-ALCAM complex.

2. The method of claim 1, wherein the monoclonal anti-CD6 is itolizumab.

3. The method of claim 1, wherein the reduction of phosphorylation of CD6 of the CD6-ALCAM complex also causes a reduction in docking of ZAP 70 and SLP-76.

4. The method of claim 1 wherein reduced phosphorylation of CD6 receptor of the CD6-ALCAM complex causes reduction in the expression of phosphatases SHP1 and SHP2.

5. The method of claim 1, wherein the host cell is in a human subject.

6. The method of claim 2, wherein the itolizumab antibody does not inhibit the binding of ALCAM to CD6 at D3 but does inhibit full interaction of the formed CD6-ALCAM complex due to steric hindrance at the immunological synapse.

7. A method inhibiting full interaction of the formed CD6-ALCAM complex due to steric hindrance at the immunological synapse, the method comprising:
contacting a host cell with a monoclonal anti-CD6 antibody comprising heavy and light chain variable regions as set forth in SEQ ID NO: 1 and 2 respectively, wherein the binding of the monoclonal anti-CD6 antibody to D1 receptor on CD6 causes a steric hindrance for interaction of ALCAM with D3 receptor of CD6, thereby causing a reduction of phosphorylation of CD6 receptor of the CD6-ALCAM complex.

8. The method of claim 7, wherein the monoclonal anti-CD6 is itolizumab.

9. The method of claim 7, wherein the reduction of phosphorylation of CD6 of the CD6-ALCAM complex also causes a reduction in docking of ZAP 70 and SLP-76.

10. The method of claim 7 wherein reduced phosphorylation of CD6 receptor of the CD6-ALCAM complex causes reduction in the expression of phosphatases SHP1 and SHP2.

11. The method of claim 7, wherein the host cell is in a human subject.

12. The method of claim 8, wherein the itolizumab antibody does not inhibit the binding of ALCAM to CD6 at D3 but does inhibit full interaction of the formed CD6-ALCAM complex due to steric hindrance at the immunological synapse.

13. A method of inhibiting expression of phosphatases SHP1 and SHP2, the method comprising:
contacting a host cell with a monoclonal anti-CD6 antibody comprising heavy and light chain variable regions as set forth in SEQ ID NO: 1 and 2 respectively, wherein the binding of the monoclonal anti-CD6 antibody to D1 receptor on CD6 causes a reduction of phosphorylation of CD6 receptor of the CD6-ALCAM complex, thereby reducing the expression of phosphatases SHP1 and SHP2.

14. The method of claim 13, wherein the monoclonal anti-CD6 is itolizumab.

15. The method of claim 13, wherein the reduction of phosphorylation of CD6 of the CD6-ALCAM complex also causes a reduction in docking of ZAP 70 and SLP-76.

16. The method of claim 13, wherein the host cell is in a human subject.

17. The method of claim 14, wherein the itolizumab antibody does not inhibit the binding of ALCAM to CD6 at D3 but does inhibit full interaction of the formed CD6-ALCAM complex due to steric hindrance at the immunological synapse.
